(19) United States
(12) Patent Application Publication
(10) Pub. No.: US 2009/0098616 A1
(43) Pub. Date: Apr. 16, 2009

(54) ENZYMATIC TREATMENT OF LIGNOCELLULOSIC MATERIALS

(76) Inventors: Murray Burke, Oakville (CA); Bradley Saville, Oakville (CA); Claudia Ishizawa, Houston, TX (US)

Correspondence Address: BERESKIN AND PARR, 40 KING STREET WEST, BOX 401, TORONTO, ON, M5H 3Y2, CA

(21) Appl. No.: 12/247,554

(22) Filed: Oct. 8, 2008

Related U.S. Application Data
(60) Provisional application No. 60/978,585, filed on Oct. 9, 2007.

(51) Int. Cl. C12P 19/14

(52) U.S. Cl. 435/99

(57) ABSTRACT

A method for treating plant materials to release fermentable sugars is disclosed. More specifically, a two-stage enzymatic hydrolysis process for treating lignocellulosic materials and producing a sugar rich process stream that may subsequently be subjected to fermentation to produce biofuels and chemicals is disclosed.

### CROSS-REFERENCE

This application is a regular application claiming priority from U.S. Provisional application 60/978,585 filed on Oct. 19, 2007.

### FIELD

This application relates to a method for treating plant materials to release fermentable sugars. More specifically, this application relates to a two-stage enzymatic hydrolysis process for treating lignocellulosic materials and producing a sugar rich process stream that may subsequently be subjected to fermentation to produce biofuels and chemicals.

### BACKGROUND

Although biomass has long shown promise as a renewable source of fuel energy, there remains a need for more efficient means of transforming biomass into suitable biofuels. Plant materials are a significant source of fermentable sugars, such as glucose that can be transformed into biofuels. However, the sugars in plant materials are contained in long polymeric chains of cellulose and hemicellulose. Utilizing current fermentation processes, it is necessary to break down these polymeric chains into monomeric sugars, prior to the fermenting step.

Methods of converting plant biomass into fermentable sugars are known in the art and in general, comprise two main steps: a pretreatment step to loosen the plant structure, and an enzymatic or chemical hydrolysis step to convert the polymeric chains of cellulose and hemicellulose into monomeric sugars. Several approaches have been used for the pretreatment step, e.g., autohydrolysis, acid hydrolysis, ammonia activation, kraft pulping, organic solvent pulping, hot water pretreatment, ammonia percolation, lime pretreatment, caustic solvent pulping, or alkali peroxide pretreatment. Each pretreatment technology has a different mechanism of action on the plant structure, inducing either physical and/or chemical modifications. However, the main objective of the pretreatment is to provide accessibility of the plant material to the enzymes. In the autohydrolysis process, the acetyl groups attached to hemicelluloses are broken down by steam and pressure releasing organic acids, e.g., acetic acid, giving the conditions for a mild acid hydrolysis process. Although a simple process, the yield of fermentable sugars is poor, in addition to the process requiring a significant amount of energy.

Jakobsen et al. (U.S. Pat. No. 5,874,274) disclose the use of a single step enzymatic process for reducing the viscosity of a plant material using xylanase and cellulase, and in particular for the treatment of wheat.

### SUMMARY

This application relates to a two-stage enzymatic process to prepare a sugar rich process stream from a feedstock derived from plant materials. The process and apparatus may result in the conversion of at least 60%, preferably more than 75% and more preferably over 90% of the cellulose and hemicelluloses to monomeric sugars. The sugar rich process stream may subsequently be subjected to fermentation to produce an alcohol stream. The alcohol stream from the fermentation stage (i.e., the raw alcohol stream) may have an ethanol content of about 3 to about 22% v/v. Optional operating ranges include about 5 to about 15% and preferably about 5 to about 22% as well as about 8 to about 12%, preferably about 8 to about 15% and more preferably about 8 to about 22% (v/v). Such alcohol concentrations may be obtained without using corn as a feedstock.

Cellulosic ethanol processes, namely processes that produce ethanol from sugars obtained by breaking down the cellulose and/or hemicellulose from non-corn plant fiber (i.e. plant fiber that excludes corn kernels), typically produce a raw alcohol stream having an ethanol content of about 2-6% v/v. With the process and apparatus described in this application, cellulose ethanol plants may produce a raw alcohol stream having a comparable alcohol concentration to that obtained by corn based ethanol plants, namely plants that produce ethanol from sugars obtained from the starch in corn. Accordingly, one advantage of the process and apparatus of this invention is that the amount of water to be removed from the raw alcohol stream to produce a fuel ethanol stream having a comparable concentration to the concentration of a product stream from a corn based ethanol plant is substantially reduced compared to current cellulosic ethanol plant technology. As a fuel ethanol stream is typically produced by distillation, the process and apparatus described here therefore results in a substantial reduction in energy required for the distillation process and, optionally, a substantial reduction in the size (i.e., the diameter) of the distillation column compared to current cellulose ethanol plant technology. Furthermore, as the ethanol concentration increases in the raw ethanol stream, the fermentation volume decreases, representing a 2 to 3 times reduction when compared to current cellulosic ethanol plant technology.

In one embodiment, the feedstock is subjected to a first enzymatic hydrolysis process to reduce the viscosity of the feedstock and produce a low viscosity effluent stream. In an embodiment, the viscosity of the low viscosity effluent stream is preferably at least about 15% lower than the initial feedstock slurry, preferably at least about 20% lower, more preferably at least about 50% lower and most preferably at least about 90%. During the first enzymatic hydrolysis, hemicellulose and cellulose are broken down, preferably to soluble oligosaccharides of sugars. During this step, it is preferred to preferentially hydrolyze the hemicelluloses instead of the celluloses (e.g., preferentially acts on the hemicellulose relative to the cellobiose in the feedstock). For example, this process step may utilize an enzyme preparation comprising hemicellulase and cellulase activities. While it will be appreciated that a suitable enzyme preparation will typically contain enzymes that may act on the cellulose, it is preferred that only a portion of the celluloses will be converted.

Subsequently, the product stream from the first enzymatic hydrolysis process, which has a lower viscosity, is subjected to a second enzymatic hydrolysis process. The second enzymatic hydrolysis process preferably utilizes enzymes to hydrolyze cellulose as well as to convert the oligosaccharides to monomeric sugars suitable for fermentation. Preferably, this second enzyme preparation comprises beta-glucosidase activities. For example, the second enzyme preparation may have an activity to convert cellulose and cellobiose to monomers and cello-oligosaccharides. In this second enzymatic hydrolysis process, it is preferred that all, or essentially all, (e.g., preferably at least about 60, more preferably at least about 75 and most preferably at least about 90%) of the remaining cellulose and hemicelluloses, and their respective oligosaccharides, are converted, to the extent desired, but preferably to the extent commercially feasible, to monomeric sugars.

Without being limited by theory, oligosaccharides, and in particular cellobiose, have an inhibitory effect on cellulase enzymes and, in particular, on endo-gluconases and cellobiohydrolases. Accordingly, in a first step, the hemicelluloses, and optionally the cellulose, are treated with enzymes to produce soluble sugars. However, the process is conducted so as not to render a substantial portion of the cellulose into monomers or dimers, such as cellobiose. While it will be appreciated that enzymatic hydrolysis will result in the production of some monomers and cellobiose, the process is conducted so as to prevent a substantial inhibition of the enzymes. Subsequently, in a second enzymatic process, the oligosaccharides are subjected to enzymatic hydrolysis to produce fermentable sugars (preferably monomers).

Preferably, the first enzyme preparation preferentially acts on the hemicellulose. In accordance with this embodiment, without being limited by theory, it is believed that in such a first enzymatic process, the hemicellulose is broken down into oligomers and monomers that are removed from the fiber as soluble compounds in an aqueous medium (preferably water). This targeted enzymatic process opens up the fiber structure by the breakdown of the hemicellulose and the removal of the lower molecular weight compounds. In this application, the term preferentially hydrolyze means that a significant portion of the enzymes that are used target the hemicelluloses instead of the celluloses, even though some of the enzymes present may still target the celluloses. Preferred preferential hydrolysis in the first stage, include hydrolyzing about 60% or more, and preferably about 85% or more, of the hemicelluloses while, preferably, hydrolyzing less than about 25%, and more preferably less than about 15% of the celluloses. The resultant more open fiber structure permits enzymes, such as cellulases, to more readily enter the fiber structure and hydrolyze the cellulose. Accordingly, the second enzymatic hydrolysis step uses enzymes that preferentially target cellulose relative to hemicellulose in the feedstock (e.g., the second enzyme preparation preferentially acts on the cellulose and cellobiose relative to xylans in the feedstock). It will be appreciated that the second enzymatic hydrolysis step may use an enzyme preparation that includes enzymes that target hemicelluloses. However, as most of the hemicelluloses may have already been treated in the first stage, a relatively large percentage of such enzymes may not be required in the second enzyme preparation.

Without being bound by theory, it is believed that during the first enzymatic hydrolysis stage, xylan is converted to soluble xylan (soluble oligomers), and to a degree, xylose, and mannan is converted to mannose. The first enzyme preparation preferentially acts upon the β-1,4 linkage of the xylose residues of xylan and the β-1,4 linkage of the mannose residues of mannan. These rates of reaction strongly parallel the viscosity reduction that is produced by this stage. Accordingly, it is believed that the enzymatic hydrolysis of the hemicellulose results, at least in part, in the viscosity reduction and may be the main factor in the viscosity reduction.

However, many commercial hemicellulase enzyme preparations also possess cellulase activity, which may also contribute to the viscosity reduction. In particular, as the hemicellulose is hydrolyzed, water is released from the fiber, in addition to the production of oligosaccharides and monomeric sugars. Moreover, this hydrolysis results in the reduction in the length of hemicellulose and cellulose polymer chains. The release of water and the reduction in molecular chain length may also be a factor in the rapid decrease in viscosity of the mixture in the reactor during the first stage of enzymatic hydrolysis.

During the first stage enzymatic hydrolysis processes, acetyl groups are removed from the hemicellulose. In an aqueous medium, these form acetic acid. Acetic acid reduces the pH of the mixture in the reactor, e.g., from about 4.9 to about 4.4. This pH reduction has an inhibitory effect on the first stage enzyme preparation. Therefore, in accordance with a preferred embodiment, acetic acid is treated or removed from the process. For example, the acetic acid may be neutralized by the addition of a neutralizing agent (e.g., urea, anhydrous ammonia, aqueous ammonia, sodium hydroxide, potassium hydroxide) and/or acetic acid may be removed from the process, such as by operating under vacuum. As acetic acid is relatively volatile, it may be drawn off by vacuum as it is produced. Further, as the first stage enzymatic process reduces the viscosity of the mixture in the reactor, the mixture is more easily induced to flow, e.g., due to stirring, and the acetic acid has a greater chance to reach the surface of the mixture and volatilize.

Further aspects and advantages of the embodiments described herein will appear from the following description taken together with the accompanying drawing.

### BRIEF DESCRIPTION OF THE DRAWINGS

For a better understanding of the embodiments described herein and to show more clearly how they may be carried into effect, reference will now be made, by way of example only, to the accompanying drawing which shows at least one exemplary embodiment, and in which:

FIG. 1 is a flow chart of the method according to a preferred embodiment that includes optional steps;

FIG. 2 is a flow chart of the method according to another embodiment that shows additional details regarding specific process steps;

FIG. 3 is a graph demonstrating the torque vs. time of a feedstock treated in accordance with an embodiment of the disclosure;

FIG. 4 is a graph demonstrating the torque vs. time of a feedstock treated in accordance with another embodiment of the disclosure; and,

FIG. 5 is a graph comparing the graphs of FIG. 3 and FIG. 4.

### DETAILED DESCRIPTION

This application relates generally to a method of treating a lignocellulosic feedstock to breakdown cellulose and hemicellulose in the feedstock into monomeric sugars such as glucose, which may be fermented to produce alcohol. The applicants have found that the use of a two-stage enzymatic hydrolysis allows for a reduction in the viscosity of the feedstock and the production of a process stream that is rich in fermentable sugars. In an optional embodiment, the applicants have found that activating and/or physically modifying the feedstock prior to the enzymatic hydrolysis process results in an increased yield of fermentable sugars in the process stream and/or a faster reaction rate. Alternately, or in addition, in another optional embodiment, the applicants have found that subjecting the lignocellulosic feedstock to an enzymatic hydrolysis process under vacuum and removing a volatile components stream from the feedstock improves the yield of fermentable sugars and the purity of the resulting sugar rich process stream.

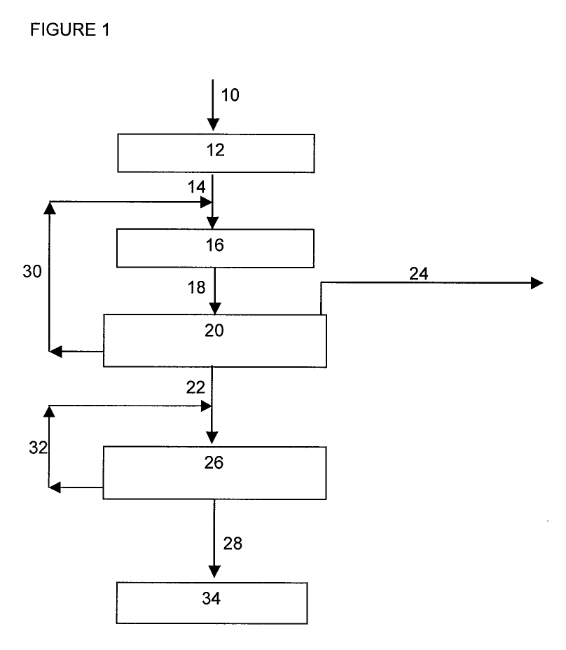

FIG. 1 exemplifies a schematic of one embodiment of the invention. The lignocellulosic feedstock 10 is optionally first subjected to activation, extraction, hydrolysis and/or physicochemical modification step 12 such as by autohydrolysis to produce an activated feedstock stream 14. The activated feedstock stream 14 is then optionally fed to a disc refiner 16 to produce a fine particulate stream 18. It will be appreciated that neither of these optional steps, or one or both of these optional steps, may be utilized.

Fine particulate stream 18 is then subjected to a two-stage enzymatic hydrolysis process. The first enzymatic hydrolysis stage 20 produces a low-viscosity effluent stream 22 and an optional volatile components stream 24, which is preferable at below atmospheric pressure in the first stage reactor 20. The low viscosity effluent stream 22 is then subject to a second enzymatic hydrolysis stage 26 to produce a sugar rich process stream 28.

All or a portion of the material subjected to a first enzymatic hydrolysis step is preferably reprocessed by recycle stream 30 and returned to the reactor 20, preferably by being passed through disc refiner 16 before being reintroduced to the first enzymatic hydrolysis stage 20. The recycle stream may be mixed with fresh lignocellulosic feedstock as exemplified prior to being introduced into the disc refiner 16. It will be appreciated that some or all of the recycle stream may be fed directly into reactor 20.

It will also be appreciated that all or a portion of the material being subjected to the second enzymatic hydrolysis step 26 is preferably removed by recycle stream 32 and returned to reactor 26.

One or both of the enzymatic hydrolysis stages may be performed under vacuum. The use of vacuum enables the production of volatile component stream 24, which can be removed from the reactor, e.g., reactor 20. The sugar rich process stream 28 may then be subjected to further processing, preferably including fermentation step 34 to produce ethanol, or it may be stored or used in other chemical processes.

### Input Feedstock

The lignocellulosic feedstock is derived from plant materials. As used herein, a “lignocellulosic feedstock” refers to plant fiber containing cellulose, hemicellulose and lignin. The applicants contemplate other sources of plant materials comprising cellulose, hemicellulose and lignin for use in deriving lignocellulosic feedstocks and any of those may be used. In some embodiments, the feedstock may be derived from trees, preferably deciduous trees such as poplar (e.g., wood chips). Alternately or in addition, the feedstock may also be derived from agricultural residues such as corn stover, wheat straw, barley straw, rice straw, switchgrass, sorghum, bagasse, rice hulls and/or corn cobs. Preferably, the lignocellulosic feedstock comprises agricultural residues and wood biomass, more preferably wood biomass and most preferably deciduous. Accordingly, the feedstock may be any feedstock that does not contain edible agricultural produce, however such material may be used.

The lignocellulosic feedstock is preferably cleaned, e.g., to remove ash, silica, metal strapping (e.g., from agricultural products), stones and dirt. The size of the components of the lignocellulosic feedstock may also be reduced. The size of the components of the feedstock may be from about 0.05 to about 2 inches, preferably from about 0.1 to about 1 inch, and more preferably from about 0.125 to about 0.5 inches in length.

It will be appreciated that if the optional activation, extraction, hydrolysis or physical modification is not utilized, the feedstock may be further crushed, ground or otherwise modified so as to decrease the average particle size of the components and increase the surface area of the material in the feedstock. Accordingly, the size of the feedstock may be from about 0.0625 to about 2 inches, preferably from about 0.125 to about 1 inch and more preferably from about 0.125 to about 0.5 inches. Any process machinery that is able to crush, grind or otherwise decrease the particle size may be utilized. The feedstock that is fed to the optional disc refiner preferably comprises from 1% to 60% wt total solids.

### Activation

The lignocellulosic feedstock is optionally subjected to one or more activation steps prior to the feedstock being subject to enzymatic hydrolysis. As used herein an “activated” feedstock refers to a feedstock that has been treated so as to increase the susceptibility of cellulose and hemicellulose in the feedstock to subsequent enzymatic hydrolysis. In addition, the lignocellulosic feedstock may also be subjected to chemical or physical modification pretreatment, extraction or hydrolysis.

The applicants have found that certain processes for treating lignocellulosic feedstocks are surprisingly beneficial for preparing the feedstocks for enzymatic hydrolysis. Without being limited by theory, the applicant's believe that activation involves the chemical activation of hydrogen bond sites in the hemicellulose and cellulose polymer chains.

Methods of activation, extraction, hydrolysis, and chemical or physical modification include, but are not limited to, autohydrolysis, acid-hydrolysis, ammonia activation, disc refining, kraft pulping, organic solvent pulping, hot water pretreatment, ammonia percolation, lime pretreatment, caustic solvent pulping and alkali peroxide pretreatment. Any process equipment known in the art may be used. Preferably, at least one of disc refining and autohydrolysis is utilized and more preferably, both are utilized.

In some embodiments, the feedstock is subjected to autohydrolysis. Autohydrolysis is a process of breaking down hemicellulose and cellulose by exposure to high temperatures, steam and pressure, preferably in the presence of a chemical agent, such as sulphuric acid. When performed in the presence of an acid, an autohydrolysis process is known as an acid hydrolysis. Autohydrolysis often results in the release of acetic acid from the breakdown of acetylated hemicellulose, which further helps the hydrolysis process.

Preferably, the autohydrolysis is conducted in a steam explosion digester, which is known in the art. For example, feedstock having a moisture content of about 45% to about 55% by weight may be fed to an autohydrolysis digester wherein the biomass is hydrolyzed under steam at high pressure (e.g. 100-400 psig) and temperature (e.g., 150-250° C.), optionally in the presence of a catalyst, such as sulphuric acid. In autohydrolysis, the acetyl groups are hydrolyzed from the plant structure producing acetic acid. The release of acetic acid decreases the pH of the reaction mixture in the digester from, e.g., neutral, to acidic (e.g., 3.0-4.0) supplying acid conditions for a mild acid hydrolysis reaction. During the autohydrolysis step, hemicellulose is partially hydrolyzed to xylose, soluble xylo-oligosaccharides and other pentosans. The yield may be up to about 75%.

During autohydrolysis, the degree of polymerization of cellulose and hemicellulose may be reduced from about 10,000 to about 1,500-1,000. This process is preferably carried out above the glass transition temperature of lignin (120-160° C.). Depending upon the severity of the reaction, degradation products may still be produced, such as furfural, hydroxyl-methylfurfural, formic acid, levulinic acid and other organic compounds.

At the instant of release from the digester (steam explosion), the biomass exits the high temperature, high pressure hydrolyzer into a reduced pressure, preferably atmospheric pressure and, more preferably into a vacuum. The pressure in the digester is suddenly released, e.g., in less than 1 second and preferably instantaneously. The rapid decrease in pressure results in the biomass separating into individual fibres or bundles of fibres. This step opens the fibre structure and increases the surface area. The lignin remains in the fibre along with cellulose and residual hemicellulose, which and then subjected to enzymatic hydrolysis for recovery of fermentable sugars from this residual cellulose and hemicellulose.

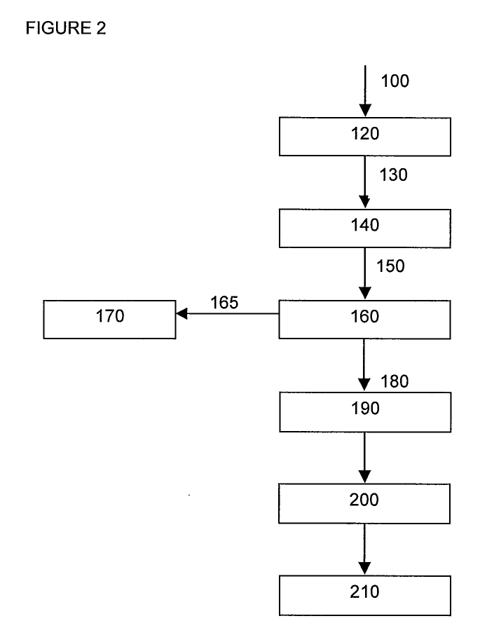

FIG. 2 exemplifies one embodiment of the invention that includes activation of the feedstock using autohydrolysis. Referring to FIG. 2, a lignocellulosic feedstock 100 is fed into a water and heat impregnator 120, where water and/or catalyst may be added to the feedstock. The addition of water is preferably carried out without steam addition to avoid the random and uncontrollable addition of moisture. The feedstock may be assayed for moisture content in order to carefully control the amount of amount water added to the feedstock. In a preferred embodiment, the moisture content of the feedstock is from about 45% to about 55% by weight before the start of autohydrolysis. The moist feedstock 130 is then subject to autohydrolysis in a hydrolyser 140. In some embodiments, the water and heat impregnation step can be performed in the same vessel as the hydrolyser.

The resulting autohydrolysed feedstock 150 may enter a solid/vapor separation unit 160 to produce a vapor stream 165 and a solid stream 180. Separation unit 160 may be operated at vacuum to remove acetic acid, furfural and other volatile compounds. The vapor stream 165 may be passed to a scrubber 170 to remove volatile products, including water, some of which may be recycled.

Still referring to FIG. 2, the resulting autohydrolyzed solid stream 180 is then preferably subjected to disc refining 190 prior to enzymatic hydrolysis 200 and fermentation 210. Any disc refiner known in the art may be used. The applicants have found that passing the chemically hydrolyzed lignocellulosic feedstock through a disc refiner further activates the feedstock and increases the susceptibility of the feedstock to enzymatic hydrolysis. The use of a disc refiner also reduces the size of the particles in the feedstock as well as increasing the total available surface area of the particles in the feedstock.

The temperature in the disc refiner is preferably maintained at less than 65° C. Above this temperature, sugar degradation may occur decreasing the sugar content in the material. Preferably, the moisture content of the fiber passing through the disc refiner is about 50 to about 99% by weight.

The applicants have found that a disc refiner can be used with a lignocellulosic feedstock at a range of different particle sizes. Preferably, the size of the particles fed to the disc refiner is from 0.0625 to 2 inches, more preferably 0.125 to 1 inch and most preferably 0.125 to 0.5 inches.

The optional use of a disc refiner prior to enzymatic hydrolysis is considered to enhance the conversion of cellulose to glucose and xylans to xylose. The use of a pulp disk refiner on an auto-hydrolyzed feedstock prior to enzymatic hydrolysis may result in an increase in the yield ratios of cellulose to glucose and xylans to xylose from about 60 to about 80% without the use of a disc refiner, to about 80 to about 95% with the use of a disc refiner.

### First Enzymatic Hydrolysis Step

The applicants herein describe a method for efficiently breaking down a lignocellulosic feedstock into fermentable sugars. Lignocellulosic feedstocks generally comprise cellulose, hemicellulose and lignin and have a high degree of polymerization. Hemicellulose is covalently linked to lignin, which in turn may be cross-linked to other polysaccharides such as cellulose resulting in a matrix of lignocellulosic material. Lignin is a hydrophobic cross-linked aromatic polymer and one of the major constituents of the cell walls of plants representing about one-quarter to one-third of the dry mass of wood.

Hemicellulose is a branched heteropolymer with a random, amorphous structure that includes a number of different sugar molecules such as xylose, glucose, mannose, galactose, rhamnose, and arabinose. Xylose is the most common sugar molecule present in hemicellulose. Xylose and arabinose are both pentosans, which are polymeric 5-carbon sugars present in plant material.

Hemicellulase enzymes break down the hemicellulose structure. The use of hemicellulase enzymes results in the breakdown of the xylan backbone and side chains into pentosans such as xylose, mannose, galactose and arabinose as well as other sugars and polysaccharides. It will be apparent to those skilled in the art that most commercial preparations of hemicellulase enzyme also possess cellulase activity. Therefore, the first enzyme preparation (i.e., a hemicellulase enzyme preparation) used in the present disclosure, may possess about 10% to about 90% hemicellulase activity, preferably about 30% to about 90% hemicellulase activity and, more preferably about 50% or more (e.g., to about 90%) hemicellulase activity. In an embodiment, the hemicellulase preferentially acts upon the β-1,4 linkage of the xylose residues of xylan and the β-1,4 linkage of the mannose residues of mannan

Cellulose is a linear polymer of glucose, wherein the glucose residues are held together by beta (1→4) glycosidic bonds. Cellulase enzymes catalyse the hydrolysis of cellulose into smaller polymeric units by breaking beta-glycosidic bonds. Endo-cellulase enzymes generally cleave internal glycosidic bonds in cellulose to create smaller polysaccharide chains, while exo-cellulase enzymes are able to cleave off 2-4 units of glucose from the ends of cellulose chains. Cellulase enzymes are not generally capable of cleaving cellulose into individual glucose molecules.

In contrast, cellobiase or beta-glucosidase enzymes catalyze the hydrolysis of a beta-glycosidic linkages resulting in the release of at least one glucose molecule. Beta-glucosidase is therefore able to cleave cellobiose, which consists of two molecules of glucose joined together by a beta-glycosidic bond.

A person skilled in the art will appreciate that enzymes may exhibit a range of different activities on different substrates. As used herein, an enzyme preparation “preferentially acts” on a substrate when the relative activity of the enzyme for that substrate is greater than for other possible substrates. For example, a hemicellulase would preferentially act on hemicellulose to produce pentosans relative to its activity for cellulose to produce glucose.

An enzyme preparation may be a single enzyme or a combination of multiple enzymes. While enzyme preparations may be isolated from a number of sources such as natural cultures of bacteria, yeast or fungi a person skilled in the art will appreciate using enzymes produced using recombinant techniques.

In some embodiments, the applicants have found that the two-stage enzymatic hydrolysis process described in the present application is able to increase the total solids content of the resulting sugar rich process stream.

As used herein, “total solids content” refers to the total amount of soluble and insoluble material in the feedstock. For example, in a lignocellulosic feedstock, soluble material would include monomeric sugars, some oligosaccharides, organic acids, extractives and low molecular weight compounds resulting from the autohydrolysis. Insoluble materials would include cellulose, lignin and hemicellulose. Suspensions with a high content of insoluble materials are generally difficult to process due to their high viscosity. Further, high-viscosity mixtures are difficult, if not impossible, to mix or handle through conventional pumping processes. In some embodiments, the sugar rich process stream described in the present application has a total solids content of greater than about 15%. In a further embodiment, the sugar rich process stream has a total solids content from about 15 to about 30%. In a further embodiment, the sugar rich process stream may have a total solids content up to about 50% (e.g., about 15 to about 50%, preferably about 30 to about 50%).

While not limited by a particular theory, the applicants note that by performing the enzymatic hydrolysis in two stages, the hemicellulase enzymes and in particular xylanase are not exposed to inhibitory concentrations of sugar monomers and dimers, and in particular glucose and cellobiose, that are produced during the second enzymatic hydrolysis stage.

The first enzymatic hydrolysis stage uses a first enzyme preparation that preferably comprises hemicellulase. As will be known by those skilled in the art, the hemicellulase preparation will also possess cellulase activity. In one embodiment, the first enzyme preparation is a xylanase enzyme cocktail such as Dyadic XBP™. In a further embodiment, the first enzyme preparation is AlternaFuel 100L™. It will be understood by a person skilled in the art that combinations of the enzyme preparations may be used. In an embodiment, the first enzyme preparation will possess hemicellulase activity from about 10% to about 90% and cellulase activity from about 90% to about 10%. In an embodiment, the hemicellulase activity will be from about 30% to about 90% and the cellulase activity will be from about 70% to about 10%. In a further embodiment, the hemicellulase activity will be from about 50% to about 90% and the cellulase activity will be from about 50 to about 10%.

In one embodiment, the pH of the process is adjusted using an acid stream or a base stream such that the pH of the feedstock is in a range suitable for enzymatic activity. In a preferred embodiment, the pH is adjusted to be between about 4.5 to about 6.0.

The temperature of the first enzymatic process may also be controlled. In one embodiment the temperature of the process is adjusted to be between about 20° C. to about 70° C. In a further embodiment, the first enzymatic process is conducted between about 30° C. to about 70° C. The process may be cooled using indirect cooling water, or warmed using indirect steam heating or by other methods known in the art.

The result of the first enzymatic process on the feedstock is a low viscosity effluent stream that may comprise xylans, cellobiose, glucose, xylose, lignin, ash, and organic acids. The low viscosity effluent stream may have a viscosity that is at least about 15% lower than that of the feedstock slurry, preferably at least about 20% lower and more preferably at least about 50% lower. Generally, the action of the first enzyme preparation results in the production of short-chain polysaccharides (oligosaccharides) such as cellobiose but not large quantities of individual glucose molecules. Without being bound by theory, this is thought to prevent the hemicellulase enzymes in the first enzyme preparation from being inhibited by glucose molecules.

In one optional embodiment, the first enzymatic process is performed under vacuum and results in a volatile components stream, which can be removed from the low viscosity effluent stream. In one embodiment, the volatile component stream includes at least one yeast, fungi, bacteria or enzyme inhibiting compound present during the first enzymatic hydrolysis process and the volatile component stream that is drawn off includes at least one inhibiting compound. In another embodiment, the inhibiting compound in the volatile component stream may contain water, acetic acid, furfural, formic acid, and any other volatile organic compounds.

### First Recycle Stream

In one embodiment, a recycle stream comprising material from the first enzymatic hydrolysis process is obtained and at least a portion of that recycle stream is preferably passed through a disc refiner or some other means of physically modifying (e.g., size reducing) the feedstock and reintroduced into the first enzymatic hydrolysis process. In an embodiment, the portion of the recycle stream that is passed through a disc refiner is between about 10 to about 90%. In another embodiment, a recycle stream from the bottom of the first enzymatic process tank is removed and is passed through a disc refiner before being reintroduced to the top of the first enzymatic process tank. The recycle stream can be mixed with fresh feedstock in the disc refiner, or prior to being reintroduced to the first enzymatic process tank.

### Second Enzymatic Hydrolysis Step

In the second enzymatic hydrolysis process, the low viscosity effluent stream is treated with a second enzyme preparation to produce a sugar rich process stream high in fermentable sugars such as glucose.

The second enzyme preparation preferably primarily includes cellulase activity. In another embodiment, the second enzyme preparation comprises beta-glucosidase activity to convert disaccharides and other small polymers of glucose into monomeric glucose. In one embodiment, the second enzyme preparation is Novozym 188™, available from Novozymes™. In another embodiment, the second enzyme preparation is NS50073™. It will be understood by those in the art that combinations of the enzyme preparations may be used.

In one embodiment, the pH of the second hydrolysis process is adjusted using an acid stream or a base stream such that the pH of the feedstock slurry is in a range suitable for enzymatic activity. In a preferred embodiment, the pH is adjusted to be between about 4.5 to about 5.4. In an embodiment, the acid stream comprises any mineral acid. In another embodiment, the acid stream comprises nitric acid, sulphuric acid, phosphoric acid, acetic acid and/or hydrochloric acid. In an embodiment, the base stream comprises potassium hydroxide, sodium hydroxide, ammonium hydroxide, urea and/or ammonia.

The temperature of the second enzymatic process may also be controlled. In one embodiment the temperature of the process adjusted to be between about 20 to about 70° C. In a further embodiment, the second enzymatic process is conducted between about 30 to about 70° C. The process may be cooled using indirect cooling water, or warmed using indirect steam heating or by other methods known in the art.

The resulting sugar rich process stream contains between about 5 to about 45% w/w fermentable sugars. Optional ranges include about 5 to about 30%, preferably about 10 to about 30% and more preferably about 15 to about 25%, as well as about 10 to about 45%, preferably about 15 to about 45% and more preferably about 25 to about 45%. The sugar rich process stream optionally also contains a total solids content of between about 10% to about 60%.

### Vacuum

The presence of certain compounds in the lignocellulosic feedstock has been found by the applicants to have an inhibitory effect on enzymatic hydrolysis and on the fermentation of the resulting sugar streams. As used herein, “inhibiting compounds” are compounds that have an inhibitory effect on the enzymatic hydrolysis process, yeast fermentation or recovery of alcohols from lignocellulosic feedstocks. Examples of inhibiting compounds include furfural, hydroxymethylfurfural, organic acids, and phenolic compounds. In a further embodiment, the inhibitory compounds are acetic acid or formic acid.

The applicants have found that performing the enzymatic hydrolysis of a lignocellulosic feedstock under vacuum allows for the removal of inhibiting compounds from the feedstock or produced during the enzymatic hydrolysis. The enzymatic hydrolysis steps are performed under vacuum so as to obtain a sugar rich process stream and a volatile components stream. In another embodiment, the volatile components stream includes at least one inhibiting compound. In one embodiment, the volatile components stream is continuously removed from the first enzymatic hydrolysis process. In a preferred embodiment, the volatile components stream is removed by performing the enzymatic hydrolysis under vacuum pressure.

In an embodiment of the disclosure, the enzymatic hydrolysis is performed under a slight vacuum. The vacuum may be from 700 to 50 mm Hg (i.e, the pressure in the vessel may be from 700 to 50 mm Hg). Preferably, the vacuum is less than about 600 mm Hg, more preferably less than about 100 mm Hg and most preferably less than about 50 mm Hg. Preferably, the maximum vacuum that is applied is about 4 mm Hg.

### OTHER EMBODIMENTS

In some embodiments, the sugar rich process stream is used to produce sugar derived products. In one embodiment of the invention, the sugar rich process stream is used to produce alcohol through fermentation. The fermentable sugars such as glucose and xylose may be fermented to alcohol after yeast addition. In an embodiment, the alcohol produced is methanol, ethanol and/or butanol.

It will be appreciated that certain features of the invention, which are, for clarity, described in the context of separate embodiments or separate aspects, may also be provided in combination in a single embodiment.

Conversely, various features of the invention, which are, for brevity, described in the context of a single embodiment or aspect, may also be provided separately or in any suitable sub-combination.

Although the invention has been described in conjunction with specific embodiments thereof, if is evident that many alternatives, modifications and variations will be apparent to those skilled in the art. Accordingly, it is intended to embrace all such alternatives, modifications and variations that fall within the spirit and broad scope of the appended claims. In addition, citation or identification of any reference in this application shall not be construed as an admission that such reference is available as prior art to the present invention.

### EXAMPLES

The operation of the invention is illustrated by the following representative examples. As is apparent to those skilled in the art, many of the details of the examples may be changed while still practicing the disclosure described herein.

### Example 1

### Viscosity Reduction Using NS50073® and Novo 188®

In a stirred reaction vessel, 2.11 mL of the cellulose enzyme preparation NS 50073™ (2.5% w/w on sugars) and 1.26 mL of the β-glucosidase enzyme preparation Novo 188™ (1.5% w/w on sugars) was added to 1.1 kg of water. The pH of the slurry was maintained at about 4.8 to about 5.2, using sodium hydroxide. The temperature was maintained at about 50° C. to about 55° C. using a metal hot water jacket and a hot water circulating bath. The reactor was then closed and agitated for about 20 minutes.

At this point, the stirring was interrupted and 356.7 g of poplar fiber (wet basis) was added to the reaction vessel, resulting in a 10% slurry consistency. The reaction vessel was then closed and set at an agitated speed of 500 RPM.

Table 1 sets out the viscosity of the feedstock slurry during the hydrolysis reaction producing a low-viscosity effluent stream measured at various times, wherein the start time is 20 minutes. Viscosity was measured by motor torque.

TABLE 1
Torque of the feedstock slurry using NS50073 ™ and Novo 188 ™

| Time | Time | | Torque (oz/in) | Torque (Nm) |
|---|---|---|---|---|
| 0 | 9:45 | am | 13.5 | 0.0953235 |
| 5 minutes | 9:50 | am | 13.3 | 0.0939113 |
| 20 minutes | 10:05 | am | 58.1 | 0.4102441 |
| 30 minutes | 10:15 | am | 46.4 | 0.3276304 |
| 45 minutes | 10:30 | am | 31.1 | 0.2195971 |
| 1 hour, 45 | 11:30 | am | 20.0 | 0.14122 |
| minutes | | | | |
| 2 hours, 20 | 12:05 | pm | 18.7 | 0.1320407 |
| minutes | | | | |
| 2 hours, 45 | 12:30 | pm | 18.0 | 0.127098 |
| minutes | | | | |
| 4 hours, 45 | 2:30 | pm | 17.2 | 0.1214492 |
| minutes | | | | |
| 5 hours, 23 | 3:08 | pm | 17.1 | 0.1207431 |
| minutes | | | | |
| 5 hours, 50 | 3:35 | pm | 17.0 | 0.120037 |
| minutes | | | | |
| 6 hours, 45 | 4:30 | pm | 16.6 | 0.1172126 |
| minutes | | | | |
| 23 hours, 45 | 9:30 | am | 15.1 | 0.1066211 |
| minutes | | | | |
| 24 hours | 9:45 | am | 15.1 | 0.1066211 |
| 24 hours, 45 | 10:30 | am | 15.0 | 0.105915 |
| minutes | | | | |
| 25 hours, 45 | 11:30 | am | 14.8 | 0.1045028 |
| minutes | | | | |
| 25 hours, 55 | 11:40 | am | 14.7 | 0.1037967 |
| minutes | | | | |
| 26 hours, 45 | 12:30 | pm | 14.7 | 0.1037967 |
| minutes | | | | |
| 28 hours, 45 | 2:30 | pm | 14.5 | 0.1023845 |
| minutes | | | | |
| 30 hours, 45 | 4:30 | pm | 14.2 | 0.1002662 |
| minutes | | | | |
| 48 hours | 9:45 | am | 13.9 | 0.0981479 |
| 48 hours, 45 | 10:30 | am | 13.8 | 0.0974418 |
| minutes | | | | |

*Conversion factor: 1 oz/in = 0.077061 Nm

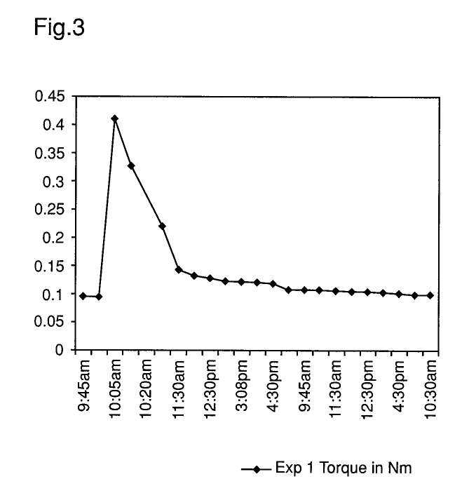

FIG. 3 shows a graphical representation of the data in Table 1, which demonstrates the viscosity reduction of a lignocellulosic feedstock (poplar fiber) slurry containing cellulose, hemicellulose and lignin. As seen in FIG. 3, the torque necessary to mix the feedstock slurry with the NS50073™ and Novo 188™ approaches 0.45 Nm (Newton meters) at the beginning of the reaction, just after the addition of the poplar fiber. The enzymes begin to lower the viscosity of the feedstock slurry immediately. It was approximately 1 hour and 45 minutes after the addition of the enzyme preparations that the viscosity had been reduced by 66%.

### Example 2

### Viscosity Reduction using NS50073™, Novo 188™ and AlternaFuel 100L™

In a stirred reaction vessel, 2.11 mL of the cellulose enzyme preparation NS 50073™ (2.5% w/w on sugars), 1.26 mL of the β-glucosidase enzyme preparation Novo 188™ (1.5% w/w on sugars) and 0.84 mL of xylanase AlternaFuel100L™ (1.0% w/w on sugars) were added to 1.1 kg of water. The pH of the slurry was maintained at about 4.8 to about 5.2, using sodium hydroxide. The temperature was maintained at about 50° C. to about 55° C. using a metal hot water jacket and a hot water circulating bath. The reactor was then closed and agitated for about 40 minutes.

At this point, the stirring was interrupted and 356.1 g of poplar fiber (wet basis) was added to the reaction vessel, resulting in a 10% slurry consistency. The reaction vessel was then closed and set at an agitated speed of 250 RPM.

Shown in Table 2 is the viscosity of the feedstock slurry during the hydrolysis reaction producing a low-viscosity effluent stream measured at various times.

TABLE 2
Torque of the feedstock slurry using NS50073 ™, Novo 188 ™ and
AlternaFuel 100L ™

| Time | | Torque (oz/in) | Torque (Nm) |
|---|---|---|---|
| 9:45 | am | 11.6 | 0.0819076 |
| 10:10 | am | 11.6 | 0.0819076 |
| 10:25 | am | 67.3 | 0.4752053 |
| 10:30 | am | 33.7 | 0.2379557 |
| 11:30 | am | 22.7 | 01602847. |
| 12:30 | pm | 20.2 | 0.1426322 |
| 1:30 | pm | 21.3 | 0.1503993 |
| 2:30 | pm | 19.2 | 0.1355712 |
| 3:30 | pm | 21.9 | 0.1546359 |
| 4:30 | pm | 19.4 | 0.1369834 |
| 9:30 | am | 16.1 | 0.1136821 |
| 10:00 | am | 16.3 | 0.1150943 |
| 10:30 | am | 15.8 | 0.1115638 |
| 11:25 | am | 15.6 | 0.1101516 |
| 11:30 | am | 12.9 | 0.0910869 |
| 12:30 | pm | 13.4 | 0.0946174 |
| 2:30 | pm | 13.0 | 0.091793 |
| 4:30 | pm | 14.2 | 0.1002662 |
| 9:10 | am | 13.2 | 0.0932052 |
| 10:30 | am | 13.0 | 0.091793 |

*Conversion factor: 1 oz/in = 0.077061 Nm

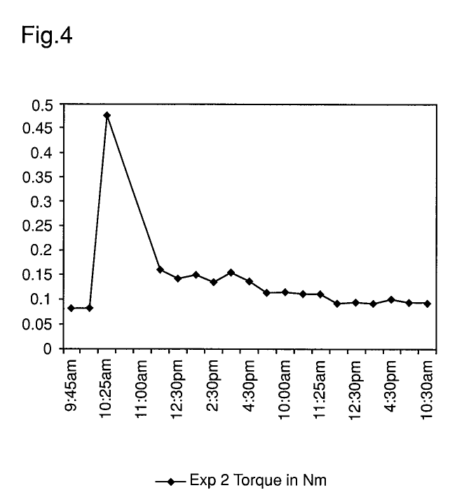

FIG. 4 shows a graphical representation of the data in Table 2, which demonstrates the viscosity reduction of a lignocellulosic feedstock (poplar fiber) slurry containing cellulose, hemicellulose and lignin. As seen in FIG. 4, the torque necessary to mix the feedstock slurry with the NS50073™, Novo 188™ and AlternaFuel 100L™ approaches 0.5 Nm (Newton meters) at the beginning of the reaction, just after the addition of the fiber. The enzymes begin to lower the viscosity of the feedstock slurry immediately. It was approximately 1.05 hours after the addition of the enzyme preparations (NS50073™, Novo 188™ and AlternaFuel 100L™) that the viscosity.

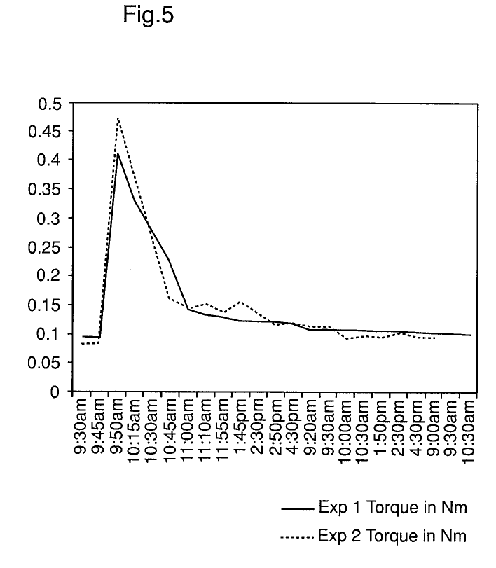

FIG. 5 is a graphical representation comparing the graphs from FIGS. 3 and 4, and graphically illustrates the increased rate of viscosity reduction of the feedstock.

1. A method for treating a lignocellulosic feedstock comprising cellulose, hemicellulose and lignin to produce a sugar rich process stream, the method comprising:
a) subjecting the feedstock to a first enzymatic hydrolysis process using a first enzyme preparation and obtaining a volatile component stream and a low viscosity effluent stream;
b) subjecting the low viscosity effluent stream to a second enzymatic hydrolysis process using a second enzyme preparation and obtaining a sugar rich process stream.

2. The method of claim 1 wherein the low viscosity effluent stream has a viscosity that is at least 15% lower than that of the feedstock.

3. The method of claim 1 wherein the low viscosity effluent stream has a viscosity that is at least 20% lower than that of the feedstock.

4. The method of claim 1 wherein the low viscosity effluent stream has a viscosity that is at least 50% lower than that of the feedstock.

5. The method of claim 1 wherein the first enzyme preparation preferentially acts upon the hemicellulose relative to cellobiose in the feedstock.

6. The method of claim 1 wherein the first enzyme preparation has hemicellulase activity and cellulase activity.

7. The method of claim 6 wherein the first enzyme preparation has a hemicellulase activity of about 10 to about 90% and a cellulase activity of between 90 to 10%.

8. The method of claim 6, wherein the first enzyme preparation has a hemicellulase activity of about 30% to about 90% and a cellulase activity of about 70% to about 10%.

9. The method of claim 8, wherein the first enzyme preparation has a hemicellulase activity of about 50% to about 90% and a cellulase activity of about 50% to 10%

10. The method of claim 7 wherein the hemicellulase enzymes preferentially act upon the β-1,4 linkage of the xylose residues of xylan and the β-1,4 linkage of the mannose residues of mannan.

11. The method of claim 1 wherein the second enzyme preparation preferentially acts on the cellulose and cellobiose relative to xylans in the feedstock.

12. The method of claim 11 wherein the second enzyme preparation comprises β-glucosidase and cellulase enzymes, wherein the β-glucosidase and cellulase enzymes preferentially act upon the β-1,4 linkage of cellobiose and cellulose.

13. The method of claim 12 wherein the p-glucosidase and cellulase enzymes completely convert cellulose and oligosaccharides produced from the first enzymatic hydrolysis to monomeric sugars.

14. The method of claim 13, wherein at least 60% of the cellulose and oligosaccharides are converted to monomeric sugars.

15. The method of claim 14, wherein at least 75% of the cellulose and oligosaccharides are converted to monomeric sugars.

16. The method of claim 15, wherein at least 90% of the cellulose and oligosaccharides are converted to monomeric sugars

17. The method of claim 1 wherein the feedstock is subjected to at least one of activation, extraction, hydrolysis and physical modification prior to step (a).

18. The method of claim 17 wherein the at least one of activation, extraction, hydrolysis and physical modification is produced by at least one of auto-hydrolysis, acid hydrolysis, ammonia activation, disc refining, kraft pulping, organic solvent pulping, hot water pretreatment, ammonia percolation, lime pretreatment, caustic solvent pulping, and alkali peroxide pretreatment.

19. The method of claim 18 wherein the at least one of activation, extraction, hydrolysis and physical modification comprises autohydrolysis that is conducted by steam explosion.

20. The method of claim 1 further comprising using the sugar rich process stream to produce sugar derived products for fermentation.

21. The method of claim 1 further comprising using the sugar rich process stream to produce a raw alcohol stream through fermentation.

22. The method of claim 21, wherein the raw alcohol stream comprises an alcohol content of about 3% to about 22% (v/v).

23. The method of claim 22, wherein the raw alcohol stream comprises an alcohol content of about 5% to about 22% (v/v).

24. The method of claim 23, wherein the raw alcohol stream comprises an alcohol content of about 8% to about 22% (v/v).

25. The method of claim 21 wherein the alcohol is at least one of methanol, ethanol and butanol.

26. The method of claim 1 wherein at least one yeast, fungi, bacteria and enzyme inhibiting compound is present during the first enzymatic hydrolysis process and the method further comprises operating the first enzymatic hydrolysis process under vacuum.

27. The method of claim 26 wherein the first enzymatic hydrolysis process is operated under a vacuum of at least 100 mm Hg.

28. The method of claim 26 wherein the method further comprises operating the second enzymatic process under vacuum.

29. The method of claim 26 wherein the at least one inhibiting compound comprises at least one of furfural, HMF, organic acids, phenolic compounds and the method further comprises operating the first enzymatic hydrolysis process under vacuum to reduce the level of at least one inhibiting compound.

30. The method of claim 26 wherein the at least one inhibiting compound comprises at least one of furfural, HMF, organic acids, phenolic compounds and the method further comprises operating the second enzymatic hydrolysis process under vacuum to reduce the level of at least one inhibiting compound.

31. The method of claim 1 further comprising passing the feedstock through a disc refiner prior to step (a).

32. The method of claim 1 further comprising obtaining a recycle stream from step (a) and reintroducing the recycle stream into the first enzymatic hydrolysis process.

33. The method of claim 32 wherein a portion of the recycle stream is first passed through a disc refiner.
